(19) United States
(12) Patent Application Publication
(10) Pub. No.: US 2015/0322519 A1
(43) Pub. Date: Nov. 12, 2015

(54) BIOMARKERS FOR PSORIASIS TREATMENT RESPONSE

(71) Applicants: Joshua MICELWEE, Boston, MA (US); Robin MOGG, North Wales, PA (US); Devan MEHROTRA, North Wales, PA (US); MERCK SHARP & DOHME CORP., Rahway, NJ (US)

(72) Inventors: Joshua McElwee, Jamaica Plain, MA (US); Robin Mogg, Chalfont, PA (US); Devan Mehrotra, Lansdale, PA (US)

(21) Appl. No.: 14/652,229

(22) PCT Filed: Dec. 19, 2013
(86) PCT No.: PCT/US13/76337

§ 371 Date: Jun. 15, 2015

Related U.S. Application Data
(60) Provisional application No. 61/745,309, filed on Dec. 21, 2012.

(51) Int. Cl. C12Q 1/68; C07K 16/24

(52) U.S. Cl. CPC C12Q 1/6883; C07K 16/244; C12Q 2600/106; C12Q 2600/156; C07K 2317/24; A61K 2039/505

(57) ABSTRACT

Single nucleotide polymorphisms (SNPs) are provided that correlate with responsiveness of psoriasis patients to treatment with a therapeutic antibody that specifically binds to the p19 subunit of IL-23. The SNPs are used as biomarkers to prospectively selecting psoriasis patients likely to benefit from treatment with antagonists of IL-23, such as an antibody that specifically binds to the p19 subunit of IL-23.

### FIELD OF THE INVENTION

The present invention relates to biomarkers for use in prospectively selecting psoriasis patients likely to benefit from treatment with antagonists of IL-23, such as an antibody that specifically binds to the p19 subunit of IL-23.

### BACKGROUND OF THE INVENTION

Psoriasis is a chronic skin disease characterized by scaling and inflammation. Greater than 5 million people are afflicted with psoriasis in the United States with as many as 250,000 new cases of psoriasis each year. Psoriasis presents in patients of all ages and almost equally in men and women. The disease exacts a heavy cost in patient suffering and medical costs. Psoriasis patients suffer physical discomfort, restricted motion of joints, reduction of manual dexterity, and emotional distress from the skin plaque formation and in some cases also experience arthritis. Annual outpatient treatment of psoriasis was estimated in 1999 to be from $1.6 to $3.2 billion, with over 1.5 million patients seen annually for this disorder by U.S. physicians.

The immune system functions to protect individuals from infective agents, e.g., bacteria, multi-cellular organisms, and viruses, as well as from cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. The immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Immune response can produce pathological consequences, e.g., when it involves excessive inflammation, as in the autoimmune disorders. See, e.g., Abbas et al. (eds.) (2000) Cellular and Molecular Immunology, W.B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med. 345:340-350.

Interleukin-12 (IL-12) is a heterodimeric molecule composed of p35 and p40 subunits. Studies have indicated that IL-12 plays a critical role in the differentiation of naïve T cells into T-helper type 1 CD4⁺ lymphocytes that secrete IFNγ. It has also been shown that IL-12 is essential for many T cell dependent immune and inflammatory responses in vivo. See, e.g., Cua et al. (2003) Nature 421:744-748. The IL-12 receptor is composed of IL-12Rβ1 and IL-12Rβ2 subunits. See Presky et al. (1996) Proc. Nat'l Acad. Sci. USA 93:14002.

Interleukin-23 (IL-23) is a heterodimeric cytokine comprised of two subunits, p19 which is unique to IL-23, and p40, which is shared with IL-12. The p19 subunit is structurally related to IL-6, granulocyte-colony stimulating factor (G-CSF), and the p35 subunit of IL-12. IL-23 mediates signaling by binding to a heterodimeric receptor, comprised of IL-23R which is unique to IL-23 receptor, and IL-12Rβ1, which is shared with the IL-12 receptor. See Parham et al. (2000) J. Immunol. 168:5699.

A number of early studies demonstrated that the consequences of a genetic deficiency in p40 in a p40 knockout (KO) mouse were more severe than those found in a p35 KO mouse. Some of these results were eventually explained by the discovery of IL-23, and the realization that the p40 KO prevents expression of not only IL-12, but also of IL-23. See, e.g., Oppmann et al. (2000) Immunity 13:715-725; Wiekowski et al. (2001) J. Immunol. 166:7563-7570; Parham et al. (2002) J. Immunol. 168:5699-708; Frucht (2002) Sci STKE 2002, E1-E3; Elkins et al. (2002) Infection Immunity 70:1936-1948.

Recent studies, through the use of p40 KO mice, have shown that blockade of both IL-23 and IL-12 is an effective treatment for various inflammatory and autoimmune disorders. Early experiments suggested a role for IL-23 in psoriasis pathogenesis. Chan et al. (2006) J. Exp. Med. 203:2577; Blauvelt (2008) J. Invest. Dermatol. 128:1064. Subsequent genetic association identified IL23R (encoding a subunit of the IL-23 receptor) and IL12B (encoding the p40 subunit of IL-23) as a susceptibility gene for psoriasis, confirming a role for IL-23 signaling in psoriasis pathogenesis. Cargill et al. (2007) Am. J. Hum. Genet. 80:273; Capon et al. (2007) Hum. Genet. 122:201. Finally, the IL-23/IL-12 antagonist antibody ustekinumab (anti-IL-12/23p40 mAb) has been approved in the U.S. and Europe for the treatment of psoriasis. Croxtall (2011) Drugs 71:1733; Yeilding et al. (2012) Ann. N.Y. Acad. Sci. 1263:1. However, the blockade of IL-12 through p40 appears to have various systemic consequences such as increased susceptibility to opportunistic microbial infections. Bowman et al. (2006) Curr. Opin. Infect. Dis. 19:245. Accordingly, treatment with antibodies that specifically bind to the unique p19 subunit of IL-23, and thus do not inhibit the activity of IL-12, are expected to be just as effective in treatment of psoriasis but without the infection or tumor risks associated with anti-p40 therapy.

Therapeutic antibodies may be used to block cytokine activity. A significant limitation in using antibodies as a therapeutic agent in vivo is the immunogenicity of the antibodies. As most monoclonal antibodies are derived from rodents, repeated use in humans results in the generation of an immune response against the therapeutic antibody. Such an immune response may range from a loss of therapeutic efficacy to a fatal anaphylactic response. Initial efforts to reduce the immunogenicity of rodent antibodies involved the production of chimeric antibodies, in which mouse variable regions were fused with human constant regions. Liu et al. (1987) Proc. Natl. Acad. Sci. USA 84:3439-43. Additional advances in the field have led to humanized antibodies, in which all of the antibody sequences other than the complementarity determining regions (CDRs) are replaced with human sequences to minimize the immunogenicity of the therapeutic antibody. Human antibodies have also been developed in which all sequences are of human origin.

Treatment of psoriasis would be delivered most efficiently if it were possible to know in advance which patients, from among those meeting standard diagnostic criteria (e.g. those with moderate to severe chronic plaque psoriasis), would respond to treatment with any particular therapeutic agent. Pre-screening would then be used to exclude from treatment those patients unlikely to benefit from the therapeutic agent in question, such as an IL-23 antagonist. Such exclusion would reduce any risk that might be associated with treatment with the therapeutic agent for those subjects unlikely to benefit, while saving money on potentially ineffective treatment. In one example, sustained virological response to treatment of hepatitis C-infected patients with peginterferon and ribavirin has been shown to be strongly associated with the homozygous C genotype at SNP rs12979860, which is in the IL28B gene. Ge et al. (2009) Nature 461:399.

The need exists for methods of pre-screening psoriasis patients for likelihood of response to treatment with IL-23 antagonists. Such methods would preferably allow exclusion of subjects for whom such IL-23 antagonists are unlikely to provide therapeutic benefit.

### SUMMARY OF THE INVENTION

The present invention meets these needs and more by providing a set of single nucleotide polymorphisms (SNPs) correlating with the responsiveness of psoriasis patients to treatment with IL-23 antagonists, such as anti-IL-23 antibodies, such as antibodies that specifically bind to the p19 subunit of human IL-23. In one aspect the invention provides methods of selecting psoriasis patients to be excluded from treatment with an IL-23 antagonist based on the likelihood that treatment will not be efficacious in those patients based on the absence of SNPs correlating to treatment response. In another aspect the invention provides methods of treatment of psoriasis patients with an IL-23 antagonist if, and only if, they are determined to have SNPs correlating to treatment response.

In various embodiments the SNPs used to categorize patients likely to respond to treatment with IL-23 antagonists are selected from the group consisting of: rs1876117; rs2048005; rs7690470; rs4106571; and rs8073229. In some embodiments a single SNP is used for the determination, whereas in other embodiments two or more SNPs are used to make the determination.

In one aspect the invention relates to selection of psoriasis patients to be excluded from treatment with an IL-23 antagonist based on their genotype at one or more of the SNPs of the present invention. In another aspect, the invention relates to treatment of a psoriasis patient with an IL-23 antagonist based on their genotype at one or more of the SNPs of the present invention.

In one embodiment, a patient is selected for treatment with an IL-23 antagonist if he or she has the T/T or T/G genotype at SNP rs1876117, but is excluded from treatment if he or she has the G/G genotype at that same SNP.

In another embodiment, a patient is selected for treatment with an IL-23 antagonist if he or she has the G/G or G/T genotype at SNP rs2048005, but is excluded from treatment if he or she has the T/T genotype at that same SNP.

In another embodiment, a patient is selected for treatment with an IL-23 antagonist if he or she has the C/C or C/T genotype at SNP rs7690470, but is excluded from treatment if he or she has the T/T genotype at that same SNP.

In another embodiment, a patient is selected for treatment with an IL-23 antagonist is he or she has the A/A or A/C genotype at SNP rs4106571, but is excluded from treatment if he or she has the C/C genotype at that same SNP.

In another embodiment, a patient is selected for treatment with an IL-23 antagonist if he or she has the A/A or A/G genotype at SNP rs8073229, but is excluded from treatment if he or she has the G/G genotype at that same SNP.

In another embodiment, a patient is excluded from treatment with an IL-23 antagonist if he or she has the G/G genotype at rs1876117 and the G/G genotype at rs8073229, but is selected for treatment if he or she has any other genotype at either of these SNPs.

In another embodiment, a patient is excluded from treatment with an IL-23 antagonist if he or she has the T/T genotype at rs2048005 and the G/G genotype at rs8073229, but is selected for treatment if he or she has any other genotype at either of these SNPs.

In some embodiments, the IL-23 antagonists include antibodies that bind to IL-23 or its receptor, including antibodies that specifically bind to the p40 or p19 subunit of IL-23, or the IL-12Rβ1 or IL-23R subunit of IL-23 receptor. In various embodiments, the method of the present invention is performed using a solution of an antibody selected from the group consisting of an anti-human IL-23p19 antibody, such as humanized antibody 13B8, including humanized 13B8-b, or anti-IL-23p40 antibodies such as ustekinumab or briakinumab, or variants of any of these three antibodies comprising the same CDR sequences, or comprising the same light chain and heavy chain variable domains.

### BRIEF DESCRIPTION OF THE DRAWINGS

FIG. 1 shows the −1×log 10 transformed p-value for each SNP used on the current GWAS study. Values circled in red were statistically significant using a false discovery rate (FDR)-based multiplicity adjustment that limits the false discovery rate to at most 5%. See Example 1.

FIGS. 2A and 2B show the percentage of patients achieving PASI 75 at week 16 after treatment indication as a function of dose of anti-IL-23p19 antibody. Plots are provided for patients having different genotypes at SNP rs1876117, as indicated. In this and all other figures herein, “MA” refers to the “minor allele” at the SNP. Data in FIG. 2A are presented for three genotype strata (each possible genotype presented separately), whereas FIG. 2B displays the same data in two genotype strata, with the heterozygotes combined with the homozygotes for the minor allele.

FIG. 3 is similar to FIG. 2, but provides results for at SNP rs2048005. Data in FIG. 3B presents data in two genotype strata, with the heterozygotes combined with the homozygotes for the major allele.

FIG. 4 is similar to FIG. 3, but provides results for at SNP rs7690470. The results for SNP rs4106571 are identical to those shown in FIG. 4.

FIG. 5 is similar to FIG. 2, but provides results for at SNP rs8073229.

FIG. 6 is similar to FIG. 2, but provides results for a composite of the data for SNPs rs1876117 (SNP1) and rs8073229 (SNP2). Plots are provided for subjects homozygous for the major alleles at both SNPs (squares), and for all other genotypes (circles).

FIG. 7 is similar to FIG. 6, but provides results for a composite of the data for SNPs rs8073229 (SNP1) and rs2048005 (SNP2). Note that the SNP designations (SNP1 and SNP2) differ from those in FIG. 6. Plots are provided for subjects homozygous for the major allele at SNP rs8073229 and homozygous for the minor allele at SNP rs2048005 (squares), and for all other genotypes (circles).

### DETAILED DESCRIPTION

All references cited herein are incorporated by reference to the same extent as if each individual publication, database entry (e.g. GenBank sequences or GeneID entries), patent application, or patent, was specifically and individually indicated to be incorporated by reference. GenBank accession numbers for nucleic acid and protein sequences referenced herein refer to the contents of the database as of the filing date of this application. Although such database entries may be subsequently modified, GenBank maintains a public record of all prior versions of the sequences as a function of date, making such database entries an unambiguous reference to a specific sequence.

In addition, incorporation by reference of any patent or published patent application is intended to incorporate the sequences in the sequence listing for that patent or published patent application. For example, incorporation by reference of patents or published patent applications disclosing antibodies that specifically bind to IL-23p19 is intended to incorporate all sequences therein, including all CDRs, CDR variants, variable domains, and light and heavy chains, in both protein and nucleic acid form.

Citation of a reference herein is not intended as an admission that the reference is pertinent prior art, nor does it constitute any admission as to the contents or date of these publications or documents.

### I. DEFINITIONS

As used herein, including the appended claims, the singular forms of words such as “a,” “an,” and “the,” include their corresponding plural references unless the context clearly dictates otherwise.

As used herein with reference to selection of subjects or patients based on their genotype at one or more SNPs, “if” is intended to mean “if and only if.” For example, when a patient is treated with an IL-23 antagonist “if” he or she has a specified genotype, it is intended that the patient not be treated if he or she does not have the specified genotype. Similarly, when a patient is excluded from treatment “if” he or she has another specified genotype, it is intended that the patient not be excluded if he or she does not have the specified other genotype. In some instances the compete phrase “if, and only if,” is included to make this meaning clear, but otherwise is implicit when “if” is used in this context.

The genotypes at each SNP are based on the sequences provided herein for those loci (SEQ ID NOs: 71-85). These sequences include “fwd” or “rev” sequences, as those terms are used in the NCBI dbSNP database, as indicated at Table 1. Minor alleles are those less prevalent in the general population than major alleles. Because there are only two alleles for each SNP of the present invention, subjects having two copies of a minor allele necessarily have no copies of the major allele, and vice versa. As used herein, a subject having “one copy” of the minor allele must have precisely one copy of the major allele, and not two. Genomes are characterized herein as the base present at each chromosome. For example, a genotype such as “A/C” refers to a heterozygous subject with the A allele on one chromosome and a C allele at the other.

Embodiments disclosed herein, and claims, that involve use of any one or more of the SNPs disclosed herein to determine whether or not to dose a psoriasis patient with an IL-23 antagonist do not preclude use of additional SNPs, including but not limited to other SNPs disclosed herein, in making the determination. Unless otherwise clear from the context, use of any SNP in a method of the present invention encompasses use of that SNP alone or in combination with other SNPs, or with any other method of evaluating whether or not to administer an IL-23 antagonist to a psoriasis patient.

As used herein, a “psoriasis patient” is a human subject who has been diagnosed with, or is suspected to have, psoriasis, whether or not the subject has ever been treated for psoriasis. Unless otherwise indicated, or clear from the context, all methods of the present invention relate to human subject and human patients, and all IL-23 antagonists are antagonists effective in humans, including but not limited to antibodies that bind to human IL-23 or its receptor.

As used herein, a PASI refers to a Psoriasis Area and Severity Index score. Reductions in PASI values reflect improvement in psoriasis in a patient. PASI Scores are defined to include a percentage improvement in PASI value over a period of time. For example, PASI 75 refers to a 75% reduction in the PASI score over the time interval, and PASI 90 represents a 90% reduction. See Example 11.

“Responder,” as used herein in a general sense, refers to subjects whose PASI value decreases (i.e. psoriasis symptoms become less severe) as a result of treatment, and “non-responder” refers to subjects whose psoriasis symptoms do not improve (become less severe) as a result of treatment. More commonly, however, responder and non-responder are used in a specific context to distinguish between subjects achieving a pre-defined quantitative improvement, such as PASI 75 over 16 weeks, from subjects that have not met the quantitative criteria.

As used herein, the term “antibody” may refer to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc., so long as they exhibit the desired biological activity.

As used herein, when referring to antibodies, the terms “binding fragment thereof” or “antigen binding fragment thereof” encompass a fragment or a derivative of an antibody that still substantially retains the ability to bind to its target. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its affinity for its target, e.g. no more than a 10-fold change in the dissociation equilibrium binding constant (K_{d}). Preferably, a binding fragment or derivative retains at least 25%, 50%, 60%, 70%, 80%, 90%, 95%, 99% or 100% (or more) of its binding affinity, although any binding fragment with sufficient affinity to exert the desired biological effect will be useful. It is also intended that, when specified, a binding fragment can include sequence variants with conservative amino acid substitutions that do not substantially alter its biologic activity.

An “IL-23 antagonist” is a molecule that inhibits the activity of IL-23 in any way. In some embodiments, an antibody or antigen binding fragment thereof of the present invention is an IL-23 antagonist that inhibits IL-23 signaling via the IL-23 receptor, for example by binding to a subunit of IL-23 or its receptor. In other embodiments an IL-23 antagonist is a small molecule or a polynucleotide, such as an antisense nucleic acid or siRNA.

“Interleukin-23 (or “IL-23”) means a protein consisting of two polypeptide subunits, p19 and p40. The sequence of the p19 subunit (also known as IL-23p19, IL23A) is provided at any of NCBI Protein Sequence Database Accession Numbers NP_{—}057668, AAH67511, AAH66267, AAH66268, AAH66269, AAH667512, AAH67513 or naturally occurring variants of these sequences. The sequence of the p40 subunit (also known as IL-12p40, IL12B) as described in any of NCBI Protein Sequence Database Accession Numbers NP_{—}002178, P29460, AAG32620, AAH74723, AAH67502, AAH67499, AAH67498, AAH67501 or naturally occurring variants of these sequences. All of these sequences are hereby incorporated by reference in their entireties.

“Interleukin-23R” or “IL-23R” means a single polypeptide chain consisting of the sequence of the mature form of human IL-23R as described in NCBI Protein Sequence Database Accession Number NP_{—}653302 (IL23R, Gene ID: 149233) or naturally occurring variants thereof. Additional IL-23R sequence variants are disclosed at WO 01/23556 and WO 02/29060. All of these sequences and documents are hereby incorporated by reference in their entireties.

“Interleukin-12Rβ1” or “IL-12Rβ1” means a single polypeptide chain consisting of the sequence of the mature form of human IL-12Rβ1 as described in NCBI Protein Sequence Database Accession Numbers NP_{—}714912, NP_{—}005526 (IL12RB1, Gene ID: 35p4) or naturally occurring variants thereof. All of these sequences and documents are hereby incorporated by reference in their entireties.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies (although these same designations, depending on the context, may also indicate the human form of a particular protein). The humanized forms of rodent antibodies will generally comprise the same CDR sequences of the parental rodent antibodies, although certain amino acid substitutions may be included to increase affinity, increase stability of the humanized antibody, or for other reasons.

Antibodies also include antibodies with modified (or blocked) Fc regions to provide altered effector functions. See, e.g., U.S. Pat. No. 5,624,821; WO 2003/086310; WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies. A longer half-life may result in less frequent dosing, with the concomitant increased convenience and decreased use of material. See Presta (2005) J. Allergy Clin. Immunol. 116:731 at 734-35.

Antibodies also include antibodies with intact Fc regions that provide full effector functions, e.g. antibodies of human isotype IgG1, which induce complement-dependent cytotoxicity (CDC) or antibody dependent cellular cytotoxicity (ADCC) in the a targeted cell. In some embodiments, the antibodies of the present invention are administered to selectively deplete cells expressing the cognate antigen from a population of cells.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding compound with a target where the association results in reduction in the normal Brownian motion of the binding compound, in cases where the binding compound can be dissolved or suspended in solution. “Binding composition” refers to a molecule, e.g. a binding compound, in combination with a stabilizer, excipient, salt, buffer, solvent, or additive, capable of binding to a target.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### II. SINGLE NUCLEOTIDE POLYMORPHISMS THAT PREDICT RESPONSIVENESS TO TREATMENT

The present invention is based on a genome-wide association study (GWAS) of patients with moderate to severe chronic plaque psoriasis who were treated with an antibody to IL-23 or a placebo. See Example 1. A statistical test was applied to ask the question of whether the dose-response association for IL-23 antagonist treatment was influenced by the patient's genotype at a given SNP locus. Genotypes were determined at approximately 800,000 SNPs, and five were identified in which the genotype correlated with responsiveness to therapy. The SNPs are listed at Table 9 (in Example 1). The results are SNPs, or combinations of SNPs, that may be used to prospectively identify those patients most likely, and least likely, to benefit from treatment with an IL-23 antagonist. Psoriasis patients unlikely to benefit from treatment with an IL-23 antagonist can be excluded from treatment, reducing both the side-effect risk to those patients and the cost of (ineffective) treatment.

Details relating to the SNPs identified in the present invention may be found at public databases and in the scientific literature, including the dbSNP database maintained by the US National Center for Biologic Information (NCBI), as would be within the skill in the art. Sequence flanking the SNPs of the present invention are provided at Table 1. Sequence are designated “rev” (reverse) or “fwd” (forward) based on their strandedness as defined in NCBI's dbSNP database. SEQ ID NOs for each SNP are provided for the sequence having both alleles (SEQ ID NOs: 71, 74, 77, 80 and 83), the sequence having the major allele (SEQ ID NOs: 72, 75, 78, 81 and 84), and the sequence having the minor allele (SEQ ID NOs: 73, 76, 79, 82 and 85).

TABLE 1
SNPs

| | | Minor | Responder | SEQ |
|---|---|---|---|---|
| SNP | Flanking Sequences (30 nt) | Allele | Genotype | ID: |
| rs1876117 | TCAGCCTGAAGAAGGGCTAATGGCCGAAAA[G/T] | T | T/T or T/G | 71, |
| (rev) | ATTTTATGTGAGTTTCACATGGGGGCATAT | | | 72, |
| | | | | 73 |
| rs2048005 | AAACAATGTAAACTCGAATAAGGATAAGCA[G/T] | T | G/G or G/T | 74, |
| (rev) | TCCAACAGATTGAAAACCAAGAAATACATT | | | 75, |
| | | | | 76 |
| rs7690470 | ATATTGAAGCAGTTAAGAAGACCCAACACA[C/T] | T | C/C or C/T | 77, |
| (rev) | GGATAATATGAATACCTGAAGAAGAAAACC | | | 78, |
| | | | | 79 |
| rs4106571/ | GAGATTTCTTGCTAATTTTCTAGATCAGAA[A/C] | C | A/A or A/C | 80, |
| (fwd) | CATTTCTTTTCTTTGTCTTTTTTTTTGTCC | | | 81, |
| | | | | 82 |
| rs8073229 | GCCACTGACTAGTCATGTGACCTCAGATAC[A/G] | A | A/A or A/G | 83, |
| (rev) | TCATTTTGTGTCCTCACCTGTTCAACAAGG | | | 84, |
| | | | | 85 |

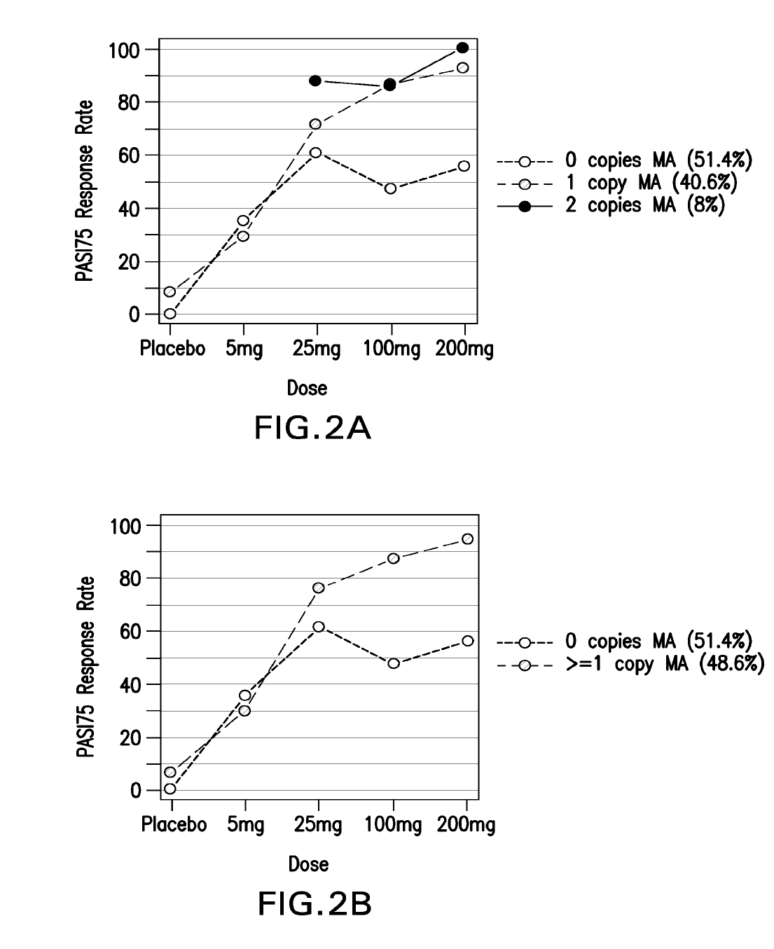

SNP rs1876117: This SNP was the single most statistically significant marker in distinguishing IL-23 antagonist responders from non-responders. About 50% of patients (G/G) had 0 copies of the minor allele and the other 50% of patients (T/T or T/G) had at least one copy of the minor allele. As illustrated in FIGS. 2A and 2B, at the two highest dose levels, patients having at least one copy of the minor allele at rs1876117 were notably more likely to achieve PASI 75 than patients homozygous for the major allele, making genotypes T/T or T/G the responders, and genotype G/G the non-responders. The dose-response curve for the major allele homozygotes was notably different from that for the minor allele homozygotes and heterozygotes when considered individually (three genotype strata), and also when compared to the combination of the heterozygotes and minor allele homozygotes (two genotype strata). See Table 2. Doses are provided in milligrams (mg). Percentages in parentheses in the “number of copies of minor allele” column represent the observed population frequency for that genotype. Three genotype strata analysis is performed by separately comparing patients with 0, 1 or 2 copies of the minor allele. Two genotype strata analysis is performed by comparing patients with 0 copies of the minor allele to patients having either 1 or 2 copies of the minor allele. See Example 3.

TABLE 2
SNP rs1876117 Results

| # copies of | PASI 75% (N evaluable) | | | | |
|---|---|---|---|---|---|
| minor allele | Placebo | 5 mg | 25 mg | 100 mg | 200 mg |
| 0 (51.4%) | 0 (19) | 35.0 (20) | 60.6 (33) | 46.9 (32) | 55.3 (38) |
| 1 (40.6%) | 8.3 (12) | 29.4 (17) | 71.4 (28) | 86.2 (29) | 92.3 (26) |
| ≧1 (48.6%) | 6.3 (16) | 29.4 (17) | 75.0 (36) | 86.1 (36) | 93.1 (29) |
| 2 (8.0%) | 0.0 (4) | (0) | 87.5 (8) | 85.7 (7) | 100.0 (3) |

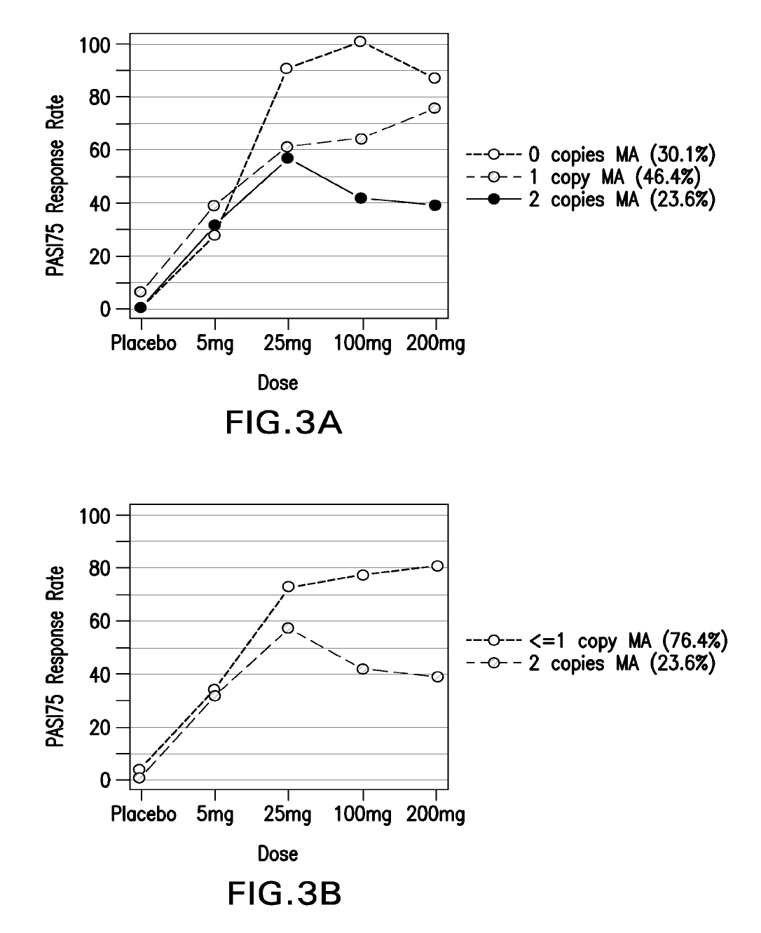

SNP rs2048005: About 75% of patients (G/G and G/T) had 0 or 1 copies of the minor allele and the other 25% of patients (T/T) had two copies of the minor allele. As illustrated in FIGS. 3A and 3B, patients having at least one copy of the major allele at rs2048005 were notably more likely to achieve PASI 75 than patients homozygous for the minor allele. Minor allele homozygotes were notably different from the major allele homozygotes and heterozygotes when considered individually (three genotype strata), and also when compared to the combination of the heterozygotes and major allele homozygotes (two genotype strata), making genotypes G/G and G/T the responders, and genotype T/T the non-responders. See Table 3, details of which are similar to those provided for Table 2, except that two genotype strata analysis is performed by comparing patients with 0 copies of the major allele to patients having either 1 or 2 copies of the major allele. See Example 4.

TABLE 3
SNP rs2048005 Results

| # copies of | PASI 75% (N evaluable) | | | | |
|---|---|---|---|---|---|
| minor allele | Placebo | 5 mg | 25 mg | 100 mg | 200 mg |
| 0 (30.1%) | 0 (12) | 27.3 (11) | 90.0 (20) | 100.0 (18) | 86.4 (22) |
| ≦1 (76.4%) | 3.4 (29) | 33.3 (24) | 71.7 (53) | 76.5 (51) | 79.6 (54) |
| 1 (46.4%) | 5.9 (17) | 38.5 (13) | 60.6 (33) | 63.6 (33) | 75.0 (32) |
| 2 (23.6%) | 0.1 (6) | 30.8 (13) | 56.2 (16) | 41.2 (17) | 38.5 (13) |

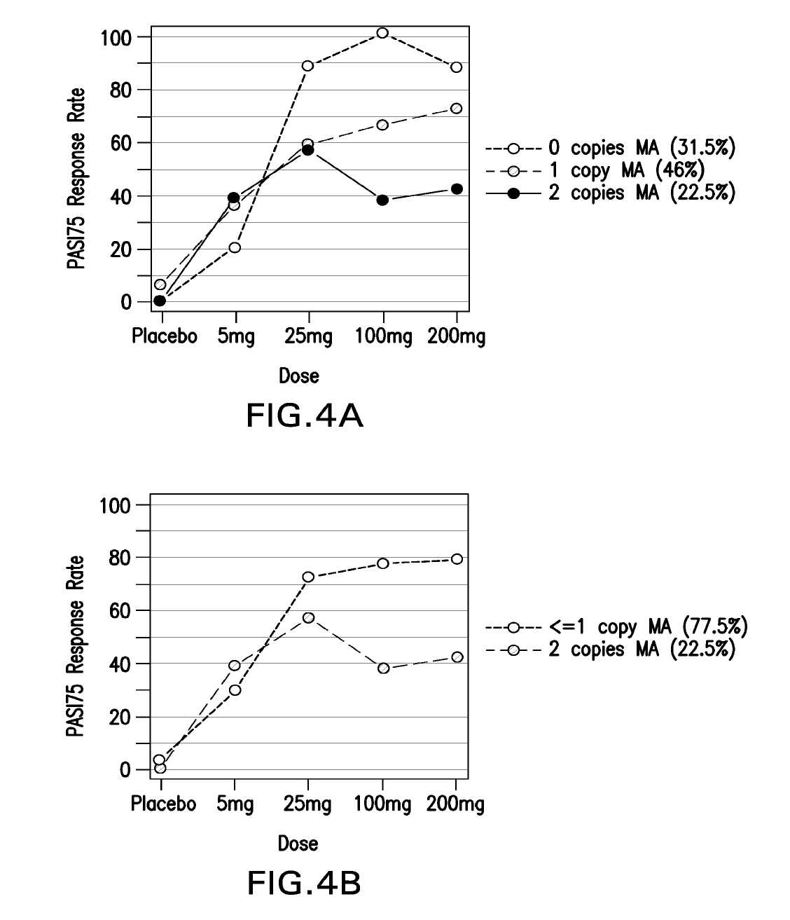

SNP rs7690470 and rs4106571: About 78% of patients had 0 or 1 copies of the minor allele (C/C or C/T) at rs7690470, and 0 or 1 copies of the minor allele (A/A or A/C) at rs4106571, and the other 22% of patients had two copies of the minor allele (T/T at rs7690470 and C/C at rs4106571). As illustrated in FIGS. 4A and 4B, patients having at least one copy of the major allele at rs7690470 or at rs4106571 were notably more likely to achieve PASI 75 than patients homozygous for the minor allele, making genotypes C/C and C/T at rs7690470 the responders and genotypes A/A and A/C at rs4106571 the responders, and genotype T/T at rs7690470 the non-responders and genotype C/C at rs4106571 the non-responders. Minor allele homozygotes were notably different from the major allele homozygotes and heterozygotes when considered individually (three genotype strata), and also when compared to the combination of the heterozygotes and major allele homozygotes (two genotype strata). See Table 4, details of which are similar to those provided for Table 3. See Examples 5 and 6.

TABLE 4
SNPs rs7690470 and rs4106571 Results

| # copies of | PASI 75% (N evaluable) | | | | |
|---|---|---|---|---|---|
| minor allele | Placebo | 5 mg | 25 mg | 100 mg | 200 mg |
| 0 (31.5%) | 0.0 (13) | 20.0 (10) | 87.5 (24) | 100.0 (17) | 87.0 (23) |
| <1 (77.5%) | 3.3 (30) | 29.3 (24) | 71.7 (53) | 76.9 (52) | 78.2 (55) |
| 1 (46.0%) | 5.9 (17) | 35.7 (14) | 58.6 (29) | 65.7 (35) | 71.9 (32) |
| 2 (22.5%) | 0.0 (5) | 38.5 (13) | 56.2 (16) | 37.5 (16) | 41.7 (12) |

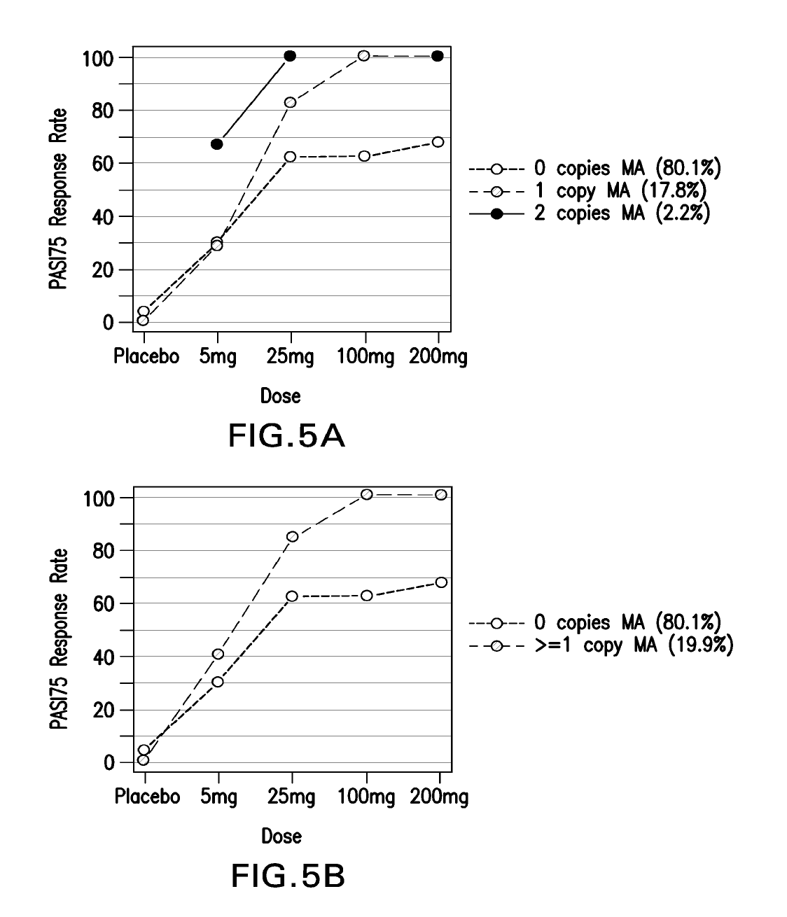

SNP rs8073229: About 80% of patients (G/G) had 0 copies of the minor allele and the other 20% of patients (A/A or A/G) had at least one copy of the minor allele. As illustrated in FIGS. 5A and 5B, the great majority of patients with at least one copy of the minor allele at SNP rs8073229 achieved PASI 75, and the percentage of patients reaching PASI 75 was notably higher than for those patients homozygous for the major allele (G/G), making genotypes A/A and A/G the responders, and genotype G/G the non-responders. However, even for patients homozygous for the major allele, the majority of the patients achieved PASI 75 at doses 25 mg or higher. Accordingly, this SNP distinguishes good responders from very good responders, and thus would be less useful in isolation as an independent marker for excluding patients from treatment with IL-23 antagonists, although it maybe useful as one component of a panel of two or more SNPs, e.g. in combination with rs1876117 or rs2048005. See Table 5, details of which are similar to those provided for Table 2. See Example 7.

TABLE 5
SNP rs8073229 Results

| # copies of | PASI 75% (N evaluable) | | | | |
|---|---|---|---|---|---|
| minor allele | Placebo | 5 mg | 25 mg | 100 mg | 200 mg |
| 0 (80.1%) | 3.6 (28) | 29.6 (27) | 62.0 (50) | 62.1 (58) | 67.2 (58) |
| 1 (17.8%) | 0.0 (7) | 28.6 (7) | 82.4 (17) | 100.0 (10) | 100.0 (8) |
| ≧1 (19.9%) | 0.0 (7) | 40.0 (10) | 84.2 (19) | 100.0 (10) | 100.0 (9) |
| 2 (2.2%) | (0) | 66.7 (3) | 100.0 (2) | (0) | 100.0 (1) |

Composite analysis can also be performed using combinations of two or more SNPs to predict patient responsiveness.

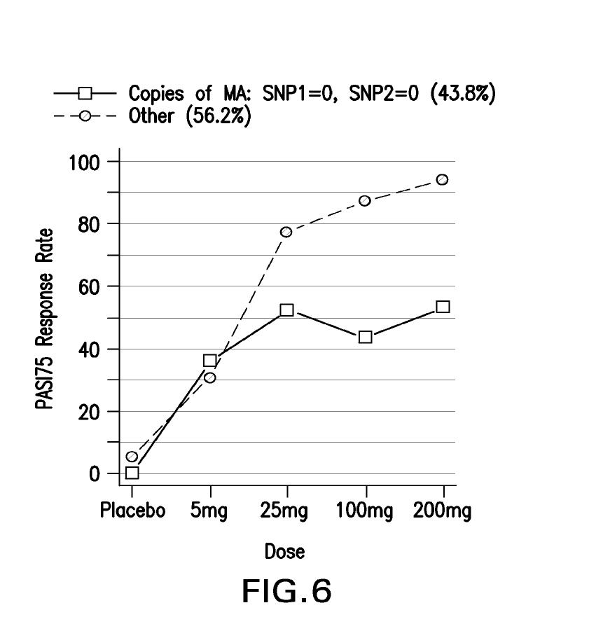

SNPs rs1876117 and rs8073229: Combining the data obtained for SNPs rs1876117 (SNP1) and rs8073229 (SNP2) it was possible to devise a composite biomarker profile for predicting the responsiveness of psoriasis patients to treatment with an IL-23 antagonist. Results are provided at Table 6. Inspection of the 100 and 200 mg dose data obtained using this composite biomarker panel (FIG. 6) shows a 40% difference in the PASI 75 response rate between patients homozygous for major alleles at both SNPs (0 copies of minor allele), who are nonresponders, and other patients, who are responders. Posterior distribution analysis indicates that there is greater than 99% probability of at least a 20% difference in response rates between these groups. See Example 8.

TABLE 6
Composite Results: SNPs rs1876117 (SNP1) and rs8073229 (SNP2)

| # copies of | PASI 75% (N evaluable) | | | | | |
|---|---|---|---|---|---|---|
| minor allele: | Population | | | | | |
| SNP1, SNP2 | Frequency | Placebo | 5 mg | 25 mg | 100 mg | 200 mg |
| 0, 0 | 43.8% | 0.0 (16) | 35.7 (14) | 52.0 (25) | 43.3 (30) | 52.8 (36) |
| 0, ≧1 | 56.2% | 5.3 (19) | 30.4 (23) | 77.3 (44) | 86.8 (38) | 93.5 (31) |
| ≧1, 0 | | | | | | |
| ≧1, ≧1 | | | | | | |

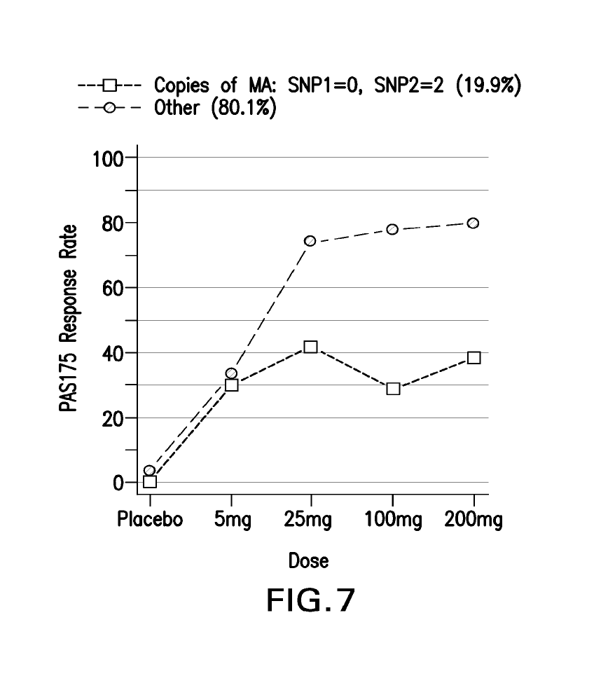

SNP rs8073229 and SNP rs2048005: Combining the data obtained for SNPs rs8073229 (SNP1) and rs2048005 (SNP2) it was possible to devise a composite biomarker profile for predicting the responsiveness of psoriasis patients to treatment with an IL-23 antagonist. Note that the SNP numbering in this section (and FIG. 7) is different from the SNP numbering for the previous section (and FIG. 6). Results are provided at Table 7. Inspection of the 100 and 200 mg dose data obtained using this composite biomarker panel (FIG. 7) shows an approximately 45% difference in the PASI 75 response rate between patients homozygous for the major allele at rs8073229 (0 copies of the minor allele) and homozygous for the minor allele at rs2048005 (2 copies of the minor allele), who are nonresponders, and patients with other genotypes, who are responders. Posterior distribution analysis indicates that there is greater than 99% probability of at least a 20% difference in response rates between these groups. It is also possible to analyze the data for this same pair of SNPs by including the heterozygotes at rs2048005 with the minor allele homozygotes, rather than with the major allele homozygotes as in Table 6, with similar results (data not shown). See Example 9.

TABLE 7
Composite Results: SNPs rs8073229 (SNP1) and rs2048005 (SNP2)

| # copies of | PASI 75% (N evaluable) | | | | | |
|---|---|---|---|---|---|---|
| minor allele: | Population | | | | | |
| SNP1, SNP2 | Frequency | Placebo | 5 mg | 25 mg | 100 mg | 200 mg |
| 0, 2 | 19.9% | 0.0 (6) | 30.0 (10) | 41.7 (12) | 28.6 (14) | 38.5 (13) |
| 0, ≦1 | 80.1% | 3.4 (29) | 33.3 (27) | 73.7 (57) | 77.8 (54) | 79.6 (54) |
| ≧1, 2 | | | | | | |
| ≧1, ≦1 | | | | | | |

Other pairwise combinations of SNPs may also be used in composite embodiments of the present invention, such as rs1876117 and rs8073229, or combinations of SNPs rs7690470 and rs4106571 with the other SNPs of the invention. These other composite biomarker set are generally less predictive (statistically) than the combinations illustrated in Tables 2 through 7, and FIGS. 6 and 7. PASI 90 data are also generally less useful than PASI 75 data in distinguishing between responders and non-responders.

### IV. IL-23 ANTAGONISTS

In general, the methods of the present invention can be used in conjunction with treatment of psoriasis patients with any IL-23 antagonist. In some embodiments the method of antagonizing IL-23 activity is a method that does not antagonize the activity of IL-12, e.g. by use of an IL-23-specific antagonist. Such methods of antagonizing IL-23 may involve blocking of the activity of the p19 subunit of IL-23, rather than the p40 subunit, since the p19 subunit is specific to IL-23 (p19+p40) whereas the p40 subunit is shared with IL-12 (p35+p40). Such methods of antagonizing IL-23 may also involve blocking of the activity of the IL-23R subunit of the IL-23 receptor complex (IL-23R+IL-12Rβ1), rather than the IL-12Rβ1 subunit that is shared with the IL-12 receptor (IL-12Rβ1+IL-12Rβ2).

In one non-limiting example, the IL-23 antagonist is an antibody that antagonizes the activity of IL-23, for example by binding to IL-23 or its receptor. Such antibodies include, but are not limited to, anti-human IL-23 antibodies (or antigen binding fragments thereof), for example an anti-human IL-23p19 antibody (or antigen binding fragment thereof) or an anti-human IL-23p40 antibody (or antigen binding fragment thereof).

In various embodiments, the anti-human IL-23p19 antibody comprises one, two, three, four, five or six of the CDR sequences, or the heavy and light chain variable domains, of the humanized antibodies disclosed in commonly assigned Int'l Pat. Appl. Pub. No. WO 2008/103432, the disclosure of which is hereby incorporated by reference in its entirety, for example antibodies hu13B8-a, -b and -c. As used herein, “hum13B8,” “hu13B8” and “h13B8” are used interchangeably to refer to humanized forms of parental mouse antibody clone 13B8, and encompasses all of forms-a, -b and -c. In another embodiment the anti-human IL-23p19 antibody competes with antibody hu13B8 for binding to human IL-23. In another embodiment the anti-human IL-23p19 antibody binds to the same epitope on human IL-23 as hu13B8. In other embodiments, the anti-human IL-23p19 antibody is able to block binding of human IL-23p19 to the antibody produced by the hybridoma deposited pursuant to the Budapest Treaty with American Type Culture Collection (ATCC—Manassas, Va., USA) on Aug. 17, 2006, under accession number PTA-7803 in a cross-blocking assay. In yet further embodiments, the anti-human IL-23p19 antibody binds to the same epitope as the antibody produced by the hybridoma deposited with ATCC under accession number PTA-7803.

Other anti-IL-23p19 antibodies that may be suitable for use in the methods of the present invention also include, but are not limited to, Eli Lilly's LY2525623 and Centocor's CNTO 1959, both of which have entered human clinical trials. Specifically, the sequences of SEQ ID NOs: 48 and 52 (heavy chain variable domains), 57 (light chain variable domain), 28-37-40 (light chain CDRs 1-2-3, respectively) and 3-8-19 (light chain CDRs 1-2-3, respectively) of EP 1937721 B1 (to Eli Lilly and Company) are hereby incorporated by reference. In addition, the sequences of SEQ ID NOs: 106 (heavy chain variable domain), 116 (light chain variable domain), 50-56-73 (light chain CDRs 1-2-3, respectively) and 5-20-44 (light chain CDRs 1-2-3, respectively) of U.S. Pat. No. 7,935,344 (to Centocor) are also hereby incorporated by reference.

Further IL-23-specific antagonists that bind to p19 include antibodies or antigen-binding fragments thereof that specifically bind to the p19 subunit of IL-23, as disclosed at WO 2007/027714, WO 2008/103432, US 2007/0048315 and WO 2008/103473 (to Schering Corp.); U.S. Pat. No. 7,491,391, U.S. Pat. No. 7,935,344 and EP 1971366 A2 (to Centocor Ortho Biotech, Inc.); WO 2007/147019, WO 2008/134659 and WO 2009/082624 (to Zymogenetics); US 2009/0311253 (to Abbott Bioresearch); and US 2009/0123479 and WO 2010/115786 (to Glaxo SmithKline), the disclosures of which are hereby incorporated by reference in their entireties.

Other exemplary IL-23-specific antagonists that bind to p19 include multimerized IL-23 receptors (US 2011/0052585 to Genzyme Corp.); protein constructs against IL-23p19 (WO 2010/142534 and WO 2009/068627 to Ablynx NV); an IL-23 aptamer (US 2006/0193821 to Archemix); and monoclonal antibody FM303 (Femta Pharmaceuticals).

In some embodiments the IL-23 antagonist is a non-specific IL-23 antagonist Exemplary non-specific IL-23 antagonists include antibodies that bind to the p40 subunit of IL-23 and IL-12, such as ustekinumab (CNTO 1275) and briakinumab (ABT-874, J-695). Ustekinumab is marketed by Centocor for the treatment of psoriasis, and is described at U.S. Pat. No. 6,902,734 and U.S. Pat. No. 7,166,285 (to Centocor, Inc.), the disclosures of which are hereby incorporated by reference in their entireties. Specifically, the sequences of SEQ ID NOs: 7 (heavy chain variable domain) and 8 (light chain variable domain), of U.S. Pat. No. 6,902,734 are hereby incorporated by reference. SEQ ID NOs: 4-5-6 and 1-2-3 of U.S. Pat. No. 6,902,734 are also incorporated by reference. Sequences for ustekinumab are also provided at SEQ ID NOs: 51-60 of the sequence listing of the present application. Briakinumab was developed by Abbott, and is described at U.S. Pat. No. 6,914,128 and U.S. Pat. No. 7,504,485, the disclosures of which are hereby incorporated by reference in their entireties. Specifically, the sequences of SEQ ID NOs: 31 (heavy chain variable domain), 32 (light chain variable domain) SEQ ID NOs; 30-28-26 (light chain CDRs 1-2-3, respectively) and 29-27-25 (heavy chain CDRs 1-2-3, respectively) of U.S. Pat. No. 6,914,128 are hereby incorporated by reference. Sequences for briakinumab are also provided at SEQ ID NOs: 61-70 of the sequence listing of the present application.

Further exemplary non-specific IL-23 antagonist antibodies that bind to the p40 subunit of IL-23 and IL-12 are disclosed at Clarke et al. (2010) mAbs 2:1-11 (Cephalon Australia, Pty., Ltd.). FM202 (Femta Pharmaceuticals) is also a monoclonal antibody that binds to the p40 subunit of both IL-12 and IL-23, as are the antibodies disclosed at WO 2010/017598 (Arana Therapeutics, Ltd.). Apilimod mesylate (STA-5326, Synta Pharmaceuticals Corp.), an oral non-specific IL-23 antagonist, may also be used in some embodiments of the present invention. Still further exemplary non-specific IL-23 antagonists include antibodies that bind to the IL-12Rβ1 subunit of both the IL-12 and IL-23 receptor complexes (WO 2010/112458 to Novartis AG).

An anti-IL-23p40-specific antibody that might be used with the methods of the present invention is disclosed in U.S. Pat. No. 7,247,711 (to Centocor), the disclosure of which is hereby incorporated by reference in its entirety.

Other IL-23 antagonists include an antibody that makes contacts with both the p19 and p40 subunits of IL-23 (WO 2011/056600 to Amgen, Inc.) and fibronectin-derived IL-23 antagonists (WO 2011/103105, developed at Adnexus Therapeutics Inc., now part of Bristol-Myers Squibb Co.).

In other embodiments the IL-23-specific antagonist binds to IL-23R. Exemplary IL-23-specific antagonists that bind to IL-23R include anti-IL-23R antibodies (WO 2008/106134 and WO 2010/027767 to Schering Corp.); multimerized and multimerized polypeptides that binds to IL-23R (U.S. Pat. App. Pub. No. 2011/0086806 to Anaphore, Inc.); and IL-23 receptor antagonist peptides (WO 2009/007849 to Valorisation HSJ and Societe en Commandite), such as APG2305 (Allostera Pharma, Inc.).

Other potential IL-23 antagonists for use in the methods of the present invention include the peptides disclosed in WO 2011/033493 (Peptinov SAS), the variant p19 polypeptides disclosed at WO 2011/011797 (Eleven Biotherapeutics, Inc. and Stanford University), and the oxidized lipid compounds disclosed at WO 2004/106486 and U.S. Pat. No. 7,625,882 (Vascular Biogenics, Ltd.), such as VBL-201 (VBL Therapeutics).

In various embodiments the IL-23 antagonist antibodies of the present invention comprise antigen binding fragments such as, but not limited to, Fab, Fab′, Fab′-SH, Fv, scFv, F(ab′)₂, and a diabody. In various other embodiments, the IL-23 antagonist is a small molecule, antisense nucleic acid, small interfering nucleic acid, aptamer, or soluble form of IL-23 receptor.

The broad scope of this invention is best understood with reference to the following examples, which are not intended to limit the inventions to the specific embodiments.

### EXAMPLES

### Example 1

### Genome-Wide Association Study—SNP Identification

Data from human subjects enrolled in a Phase IIb dose-ranging clinical trial for treatment of psoriasis were used in a genome-wide association study to identify SNPs (or groups of SNPs) correlating with responsiveness, as follows. The Phase IIb trial involved administration of an antibody that specifically binds to the p19 subunit of human IL-23 (hum13B8-b), or a placebo, to adult patients with moderate to severe chronic plaque psoriasis. Patients were dosed with drug (0, 5, 25, 100 or 200 mg) by subcutaneous injection at day 0, and four weeks thereafter. Subjects were evaluated periodically after initiation of randomized study treatment. The primary/secondary focus was on the proportion of subjects achieving a 75%/90% reduction from baseline in their PASI score at week 16 (PASI75/PASI90).

A group of 276 subjects provided appropriate genetic consent for use in the GWAS study among the overall study population of 352 subjects. The 276 patient subset had characteristics similar to the overall population, as illustrated at Table 8.

TABLE 8
GWAS Population Characteristics

| | | Overall Study | GWAS |
|---|---|---|---|
| | | Population | Population |
| Baseline Characteristic | | (N = 352) | (N = 276) |
| Randomized | Placebo | 45 (13%) | 35 (13%) |
| group: | 5 mg | 42 (12%) | 37 (13%) |
| | 25 mg | 90 (26%) | 69 (25%) |
| | 100 mg | 89 (25%) | 68 (25%) |
| | 200 mg | 86 (24%) | 67 (24%) |
| Weight ≦ 90 kg | | 222 (63%) | 165 (60%) |
| Prior Biologics Exposure | | 76 (22%) | 65 (24%) |
| Psoriatic Arthritis | | 63 (18%) | 53 (19%) |
| Race: | Asian | 59 (17%) | 30 (11%) |
| | Black or African | 4 (1%) | 4 (1%) |
| | American | | |
| | White | 288 (82%) | 242 (88%) |

| | | |
|---|---|---|
| Baseline PASI, mean (SD) | 19.6 (7.3) | 19.3 (7.4) |
| Psoriasis Duration in years, | 17 (1-52) | 17 (1-52) |
| median (range) | | |
| Age, median (range) | 45 (18-82) | 45 (18-82) |

Approximately 70% of patients achieved PASI 75 at doses of 25 mg or higher. Approximately 50% of patients achieved PASI 90 at the 200 mg dose. Unless otherwise indicated, the results presented herein are derived from PASI 75 data at 16 weeks. Results were essentially the same between the GWAS patients and the overall study population, demonstrating that the GWAS-selected subjects were representative of the overall study population.

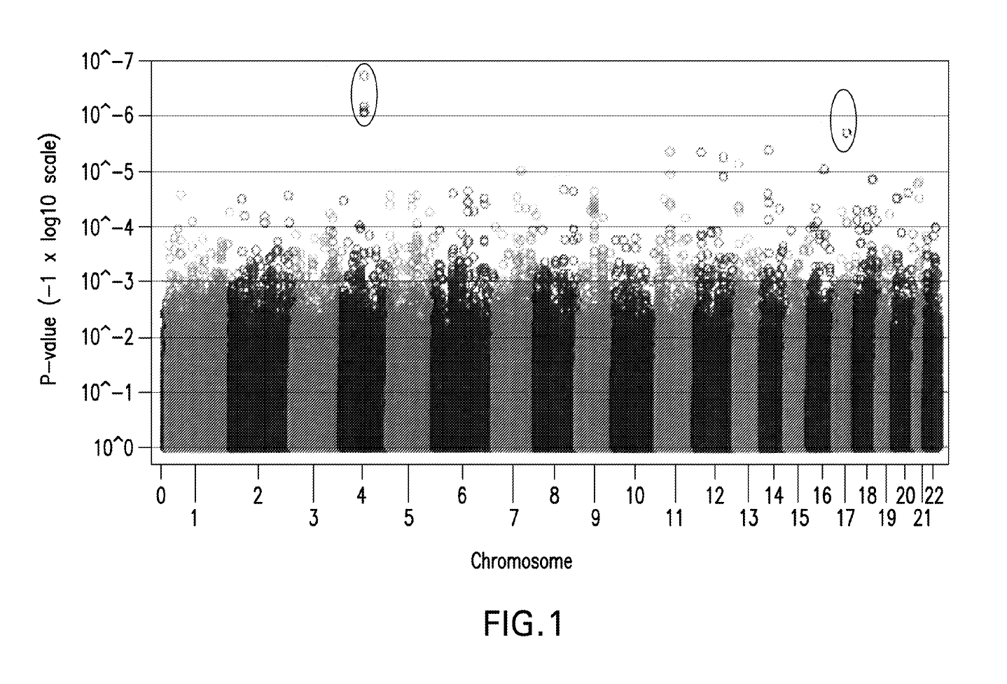

Five SNPs were identified, out of approximately 800,000 SNPs that were considered in the analysis, with statistically significant association with drug responsiveness. See FIG. 1. Four of the identified SNPs were on chromosome 4 and one was on chromosome 17. The five SNPs were statistically significant using a multiplicity adjustment (Hu et al. (2010) J. Am. Stat. Soc. 105:1215), which limits the false discovery rate (FDR) to at most 5%. The null hypothesis is that the observed dose-response was not influenced by the genotype at the SNP. The identified SNPs are provided at Table 9.

TABLE 9
Single Nucleotide Polymorphisms

| Identifier | Chromosome | Function | P-value |
|---|---|---|---|
| rs1876117 | 4 | non-coding | 1.8E−07 |
| rs2048005 | 4 | non-coding | 6.5E−07 |
| rs7690470 | 4 | non-coding | 8.2E−07 |
| rs4106571 | | | |
| rs8073229 | 17 | integrin beta3 | 1.9E−06 |

The four SNPs on chromosome 4 are tightly grouped together, and the closest gene is approximately 0.5 megabases away. The SNP on chromosome 17 is in the integrin beta3 gene, which has previously been associated with asthma and allergy (e.g. Thompson et al. (2007) J. Allergy Clin. Immunol. 119:1423), but not psoriasis. The statistical significance (p-values) of the identified SNPs did not change appreciably after statistical model-based adjustments for relevant covariates such as race, weight, and prior biologic therapy (data not shown).

### Example 2

### Anti-IL-23p19 mAb hum13B8-b

The Phase IIb dose-ranging clinical trial involved intravenous administration of the humanized anti-human Il-23p19 antibody hum13B8-b. This antibody is described in greater detail, e.g., at U.S. Pat. No. 8,293,883. The light chain complementarity determining regions (CDRs) are provided at SEQ ID NOs: 36 (CDRL1), 41 (CDRL2) and 46 (CDRL3), and the heavy chain CDRs are provided at SEQ ID NOs: 19 (CDRH1), 25 (CDRH2) and 31 (CDRH3). The light and heavy chain variable domains are provided at residues 1-108 of SEQ ID NO: 14 and residues 1-116 of SEQ ID NO: 7, respectively. The full-length light and heavy chains are provided at SEQ ID NOs: 14 and 7, respectively.

### Example 3

### Patient Selection Using SNP Rs1876117

Psoriasis patients may be selected for treatment with an IL-23 antagonist, or excluded from treatment with an IL-23 antagonist, as follows. Candidate patients are selected from among psoriasis patients meeting general criteria for treatment with the particular IL-23 antagonist in question, such as disease severity, comorbidity or prior failed treatment. For example, anti-IL-23p40 antibody ustekinumab is indicated for use in adult (18 years or older) psoriasis patients with moderate to severe plaque psoriasis who are candidates for phototherapy or systemic therapy.

Samples are obtained from candidate psoriasis patients and used to determine their genotype at SNP rs1876117. Candidates with the genotype rs1876117 (T/T or T/G) are selected for treatment with the IL-23 antagonist, whereas candidates with the genotype rs1876117 (G/G) are not treated with the IL-23 antagonist.

### Example 4

### Patient Selection Using SNP Rs2048005

Candidate psoriasis patients are selected for treatment with an IL-23 antagonist, or for exclusion from treatment with the IL-23 antagonist, as described in Example 3, except that SNP rs2048005 is used rather than SNP rs1876117. Candidates with the genotype rs2048005 (G/G or G/T) are selected for treatment with the IL-23 antagonist, whereas candidates with the genotype rs2048005 (T/T) are not treated with the IL-23 antagonist.

### Example 5

### Patient Selection Using SNP Rs7690470

Candidate psoriasis patients are selected for treatment with an IL-23 antagonist, or for exclusion from treatment with the IL-23 antagonist, as described in Example 3, except that SNP rs7690470 is used rather than SNP rs1876117. Candidates with the genotype rs7690470 (C/C or C/T) are selected for treatment with the IL-23 antagonist, whereas candidates with the genotype rs7690470 (T/T) are not treated with the IL-23 antagonist.

### Example 6

### Patient Selection Using SNP Rs4106571

Candidate psoriasis patients are selected for treatment with an IL-23 antagonist, or for exclusion from treatment with the IL-23 antagonist, as described in Example 3, except that SNP rs4106571 is used rather than SNP rs1876117. Candidates with the genotype rs4106571 (A/A or A/C) are selected for treatment with the IL-23 antagonist, whereas candidates with the genotype rs4106571 (C/C) are not treated with the IL-23 antagonist.

### Example 7

### Patient Selection Using SNP Rs8073229

Candidate psoriasis patients are selected for treatment with an IL-23 antagonist, or for exclusion from treatment with the IL-23 antagonist, as described in Example 3, except that SNP rs8073229 is used rather than SNP rs1876117. Candidates with the genotype rs8073229 (A/A or A/G) are selected for treatment with the IL-23 antagonist, whereas candidates with the genotype rs8073229 (G/G) are not treated with the IL-23 antagonist.

### Example 8

### Patient Selection Using SNPs Rs1876117 and Rs8073229

Candidate psoriasis patients are selected for treatment with an IL-23 antagonist, or for exclusion from treatment with the IL-23 antagonist, essentially as described in Examples 3 and 7, except that SNPs rs1876117 and rs8073229 are used in combination, rather than separately. Candidates with the genotype rs1876117 (G/G) and rs8073229 (G/G) are not treated with the IL-23 antagonist, whereas candidates with the other genotypes at one or both of these two SNPs are selected for treatment with the IL-23 antagonist.

### Example 9

### Patient Selection Using SNPs Rs8073229 and Rs2048005

Candidate psoriasis patients are selected for treatment with an IL-23 antagonist, or for exclusion from treatment with the IL-23 antagonist, essentially as described in Examples 4 and 7, except that SNPs rs8073229 and rs2048005 are used in combination, rather than separately. Candidates with the genotype rs8073229 (G/G) and rs2048005 (T/T) are not treated with the IL-23 antagonist, whereas candidates with the other genotypes at one or both of these two SNPs are selected for treatment with the IL-23 antagonist.

### Example 10

### Methods of Determining Genotype at SNP Loci

The genotype of a patient at a given SNP locus may be determined by any suitable method of SNP genotyping known in the art, or hereafter developed. Such methods include, but are not limited to, hybridization-based approaches, including dynamic allele-specific hybridization, molecular beacons, and SNP microarray hybridization; enzyme-based methods, including allele-selective PCR amplification (such as tetra-primer ARMS-PCR), using Flap endonuclease (such as an “Invader assay”), primer extension, an oligonucleotide ligation assay; and other methods, including single strand conformational polymorphism analysis, temperature gradient electrophoresis (TGGE), and denaturing HPLC. Alternatively, with advances in DNA sequencing technology, it may be practical to inspect whole or partial genome sequence for a subject at a given locus to determine which allele is present.

Primers and probes for use in methods to determine the genotype at the SNPs of the present invention may be derived from the sequences surrounding the SNPs, which are provided at SEQ ID NOs: 71-85.

### Example 11

### PASI Scores

PASI scores, which range from 0 to 72 (maximal disease), are determined by methods well known to those in the art of treatment of psoriasis. Fredriksson & Pettersson (1978) Dermatologica 157:238. Briefly, a medical practitioner, such as a physician, preferably a dermatologist, evaluates the percentage of total skin area of a subject affected by psoriasis in each of 4 segments of the body—the head/neck, arms, trunk, and legs. Each area percentage is converted to a “grade” based on the following conversion: 0% involved area is grade 0; <10% involved area is grade 1; 10-29% involved area is grade 2; 30-49% involved area is grade 3; 50-69% involved area is grade 4; 70-89% involved area is grade 5; and 90-100% involved area is grade 6.

Each area is then independently scored for severity of erythema (redness), induration (thickness) and desquamation (scaling) on a scale from 0 (none), 1 (mild), 2 (moderate), 3 (severe), to 4 (very severe/maximum). One of skill in the art, such as a dermatologist, will be able to assign a severity score on this 0-4 scale based on personal experience and in light of exemplary photographs. The three severity values are added for each segment of the body, and multiplied by the “grade” for that segment. The resulting value is then multiplied by a weighting factor for the relative area of the segment of the body; 0.1 for the head/neck, 0.2 for the arms, 0.3 for the trunk, and 0.4 for the legs. Resulting values for all four segments are added together to generate a PASI score. Alternative orders of mathematical steps can be used, provided the overall PASI calculation is mathematically equivalent.

A PASI score less than or equal to 10 is considered to be mild disease, whereas a score of greater than 10 is considered to be moderate to severe disease.

Decreases in PASI score are reported as a percent reduction, e.g. a PASI 75 value represents a 75% decrease in PASI score, for example after therapy as compared to the PASI score prior to initiation of therapy.

Table 10 provides a brief description of the sequences in the sequence listing.

TABLE 10
Sequence Identifiers

| SEQ ID NO: | Description |
|---|---|
| 1 | m1A11 V_{H} |
| 2 | m11C1 V_{H} |
| 3 | m5F5 V_{H} |
| 4 | m21D1 V_{H} |
| 5 | m13B8 V_{H} |
| 6 | hum13B8 HC-a |
| 7 | hum13B8 HC-b |
| 8 | hum13B8 HC-c |
| 9 | m1A11 V_{L} |
| 10 | m11C1 V_{L} |
| 11 | m5F5 V_{L} |
| 12 | m21D1 V_{L} |
| 13 | m13B8 V_{L} |
| 14 | hum13B8 LC |
| 15 | m1A11 CDRH1 |
| 16 | m11C1 CDRH1 |
| 17 | m5F5 CDRH1 |
| 18 | m21D1 CDRH1 |
| 19 | m13B8 CDRH1 |
| 20 | m1A11 CDRH2 |
| 21 | m11C1 CDRH2 |
| 22 | m5F5 CDRH2 |
| 23 | m21D1 CDRH2 |
| 24 | m13B8 CDRH2-a |
| 25 | h13B8 CDRH2-b |
| 26 | h13B8 CDRH2-c |
| 27 | m1A11 CDRH3 |
| 28 | m11C1 CDRH3 |
| 29 | m5F5 CDRH3 |
| 30 | m21D1 CDRH3 |
| 31 | m13B8 CDRH3 |
| 32 | m1A11 CDRL1 |
| 33 | m11C1 CDRL1 |
| 34 | m5F5 CDRL1 |
| 35 | m21D1 CDRL1 |
| 36 | m13B8 CDRL1 |
| 37 | m1A11 CDRL2 |
| 38 | m11C1 CDRL2 |
| 39 | m5F5 CDRL2 |
| 40 | m21D1 CDRL2 |
| 41 | m13B8 CDRL2 |
| 42 | m1A11 CDRL3 |
| 43 | m11C1 CDRL3 |
| 44 | m5F5 CDRL3 |
| 45 | m21D1 CDRL3 |
| 46 | m13B8 CDRL3 |
| 47 | human IL-23p19 |
| 48 | mouse IL-23p19 |
| 49 | hum13B8-b HC DNA |
| 50 | hum13B8 LC DNA |
| 51 | ustekinumab CDRH1 |
| 52 | ustekinumab CDRH2 |
| 53 | ustekinumab CDRH3 |
| 54 | ustekinumab CDRL1 |
| 55 | ustekinumab CDRL2 |
| 56 | ustekinumab CDRL3 |
| 57 | ustekinumab V_{H} |
| 58 | ustekinumab V_{L} |
| 59 | ustekinumab HC |
| 60 | ustekinumab LC |
| 61 | briakinumab CDRH1 |
| 62 | briakinumab CDRH2 |
| 63 | briakinumab CDRH3 |
| 64 | briakinumab CDRL1 |
| 65 | briakinumab CDRL2 |
| 66 | briakinumab CDRL3 |
| 67 | briakinumab V_{H} |
| 68 | briakinumab V_{L} |
| 69 | briakinumab HC |
| 70 | briakinumab LC |
| 71 | rs1876117 SNP |
| 72 | rs1876117 major allele (G) |
| 73 | rs1876117 minor allele (T) |
| 74 | rs2048005 SNP |
| 75 | rs2048005 major allele (G) |
| 76 | rs2048005 minor allele (T) |
| 77 | rs7690470 SNP |
| 78 | rs7690470 major allele (C) |
| 79 | rs7690470 minor allele (T) |
| 80 | rs4106571 SNP |
| 81 | rs4106571 major allele (A) |
| 82 | rs4106571 minor allele (C) |
| 83 | rs8073229 SNP |
| 84 | rs8073229 major allele (G) |
| 85 | rs8073229 minor allele (A) |

1. A method of determining whether a psoriasis patient should be excluded from treatment with an antagonist of IL-23 comprising:
a) detecting which genotypes are present at one or more SNPs in the genome of the patient, wherein the SNPs are selected from the group consisting of:
i) SNP rs1876117;
ii) SNP rs2048005;
iii) SNP rs7690470;
iv) SNP rs4106571; and
v) SNP rs8073229,
and
b) excluding the patient from treatment with said IL-23 antagonist if (and only if) the patient has at least one genotype selected from the group consisting of:
i) rs1876117 (G/G);
ii) rs2048005 (T/T);
iii) rs7690470 (T/T);
iv) rs4106571 (C/C); and
v) rs8073229 (G/G).

2. The method of claim 1 wherein the determining whether a psoriasis patient should be excluded from treatment with an antagonist of IL-23 comprises:
a) detecting which genotype is present at SNP rs1876117 in the genome of the patient; and
b) excluding the patient from treatment with said IL-23 antagonist if (and only if) the genotype is rs1876117 (G/G).

3. The method of claim 1 wherein the determining whether a psoriasis patient should be excluded from treatment with an antagonist of IL-23 comprises:
a) detecting which genotype is present at SNP rs2048005 in the genome of the patient; and
b) excluding the patient from treatment with said IL-23 antagonist if (and only if) the genotype is rs2048005 (T/T).

4. The method of claim 1 wherein the determining whether a psoriasis patient should be excluded from treatment with an antagonist of IL-23 comprises:
a) detecting which genotype is present at SNP rs7690470 in the genome of the patient; and
b) excluding the patient from treatment with said IL-23 antagonist if (and only if) the genotype is rs7690470 (T/T).

5. The method of claim 1 wherein the determining whether a psoriasis patient should be excluded from treatment with an antagonist of IL-23 comprises:
a) detecting which genotype is present at SNP rs4106571 in the genome of the patient; and
b) excluding the patient from treatment with said IL-23 antagonist if (and only if) the genotype is rs4106571 (C/C).

6. The method of claim 1 wherein the determining whether a psoriasis patient should be excluded from treatment with an antagonist of IL-23 comprises:
a) detecting which genotype is present at SNP rs8073229 in the genome of the patient; and
b) excluding the patient from treatment with said IL-23 antagonist if (and only if) the genotype is rs8073229 (G/G).

7. The method of claim 1 wherein the determining whether a psoriasis patient should be excluded from treatment with an antagonist of IL-23 comprises:
a) detecting which genotype is present at SNPs rs1876117 and rs8073229 in the genome of the patient; and
b) excluding the patient from treatment with said IL-23 antagonist if (and only if) the genotype is rs1876117 (G/G) and rs8073229 (G/G).

8. The method of claim 1 wherein the determining whether a psoriasis patient should be excluded from treatment with an antagonist of IL-23 comprises:
a) detecting which genotype is present at SNPs rs2048005 and rs8073229 in the genome of the patient; and
b) excluding the patient from treatment with said IL-23 antagonist if (and only if) the genotype is rs2048005 (T/T) and rs8073229 (G/G).

9. A method of treating a psoriasis patient with an antagonist of IL-23, comprising:
a) detecting which genotypes are present at one or more selected SNPs in the genome of the patient, wherein the SNPs are selected from the group consisting of:
i) SNP rs1876117;
ii) SNP rs2048005;
iii) SNP rs7690470;
iv) SNP rs4106571; and
v) SNP rs8073229,
and
b) administering a therapeutically effective amount of the IL-23 antagonist to the patient if (and only if) the patient has at least one genotype selected from the group consisting of:
i) rs1876117 (T/T or T/G);
ii) rs2048005 (G/G or G/T);
iii) rs7690470 (C/C or C/T);
iv) rs4106571 (A/A or A/C); and
v) rs8073229 (A/A or A/G).

10. The method of claim 9 wherein the treating a psoriasis patient with an antagonist of IL-23 comprises:
a) detecting which genotype is present at SNP rs1876117 in the genome of the patient; and
b) administering a therapeutically effective amount of the IL-23 antagonist to the patient if (and only if) the genotype is rs1876117 (T/T or T/G).

11. The method of claim 9 wherein the treating a psoriasis patient with an antagonist of IL-23 comprises:
a) detecting which genotype is present at SNP rs2048005 in the genome of the patient; and
b) administering a therapeutically effective amount of the IL-23 antagonist to the patient if (and only if) the genotype is rs2048005 (G/G or G/T).

12. The method of claim 9 wherein the treating a psoriasis patient with an antagonist of IL-23 comprises:
a) detecting which genotype is present at SNP rs7690470 in the genome of the patient; and
b) administering a therapeutically effective amount of the IL-23 antagonist to the patient if (and only if) the genotype is rs7690470 (C/C or C/T).

13. The method of claim 9 wherein the treating a psoriasis patient with an antagonist of IL-23 comprises:
a) detecting which genotype is present at SNP rs4106571 in the genome of the patient; and
b) administering a therapeutically effective amount of the IL-23 antagonist to the patient if (and only if) the genotype is rs4106571 (A/A or A/C).

14. The method of claim 9 wherein the treating a psoriasis patient with an antagonist of IL-23 comprises:
a) detecting which genotype is present at SNP rs8073229 in the genome of the patient; and
b) administering a therapeutically effective amount of the IL-23 antagonist to the patient if (and only if) the genotype is rs8073229 (A/A or A/G).

15. The method of claim 1, wherein the IL-23 antagonist is an antibody that binds to IL-23.

16. The method of claim 15 wherein the IL-23 antagonist is an antibody, or antigen binding fragment thereof, that binds to the p19 subunit of IL-23.

17. The method of claim 16 wherein the IL-23 antagonist is antibody hum13B8-b, or an antigen binding fragment thereof.

18. The method of claim 15 wherein the IL-23 antagonist is an antibody that binds to the p40 subunit of IL-23.

19. The method of claim 18 wherein the IL-23 antagonist is:
a) ustekinumab; or
b) briakinumab.


SEQUENCE LISTING (85 sequences)

SEQ ID NO: 1 - protein, 117 aa, Mus musculus
EVQLQQSGPELVKTGASVNISCKASGYSFTAYYIQWVKQSRGKSLEWIGYISCYNGATRYNQKFKGKATFTVDTSSRTAYMQFSSLTSEDSAVYFCARQGFYAMDYWGQGTSVTVSS

SEQ ID NO: 2 - protein, 116 aa, Mus musculus
HVQLQQSGPEVVRPGASVKLSCKASGYIFSAYWMTWVKQRPGQGLEWIGQIFPVRGSADYNEIFEGKATLTVDTSSSTAYIQLSSLTSEDSAVYYCARGGGGFAYWGQGTLVTVSA

SEQ ID NO: 3 - protein, 124 aa, Mus musculus
QVQLQQSGAELARPGASVKLSCKASGYTFTSYGISWVKQRTGQDLEWIGEIYPRSVNSYYNERFKGKATLTADKSSSTAYMELRSLTSEDSAVYFCARGGNYYGRNYGDYFDYWGQGTTLTVSS

SEQ ID NO: 4 - protein, 116 aa, Mus musculus
QVQLQQSGLELVKPGSSLKISCKASGYSFTSFFIHWLKQRPGQGLEWIGWIFPGNHDVEYNEKFKGKATLTADTSSSTADMHLSSLTSEDSAVYFCARGGGNLPYWGQGTLVTVSA

SEQ ID NO: 5 - protein, 116 aa, Mus musculus
HVQLQQSGPELVRPGASVELSCKASGYIFITYWMTWMKQRPGQGLEWIGQIFPASGSADYNEMFEGKATLTVDTSSNTAYMQLSSLTSEDSAVYFCARGGGGFAYWGQGTLVTVSA

SEQ ID NO: 6 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYNEMFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYAQKLQGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 113 aa, Mus musculus
NVVMTQTPLTLSVTIGQPASISCKSSQSLLDSDGKTYLNWLLQRPGQSPKRLIYLVSKLDSGVPDRFTGSGSGTDFTLKISRVEAEDLGLYYCWQGTHFPFTFGSGTKLEIKR

SEQ ID NO: 10 - protein, 108 aa, Mus musculus
DIQMTQSPASLSASVGETVTITCRASENIYSYLAWYQEKWGKSPQLLVYNAKTLAEGVPSRFSGSGSGTQFSLKINSLQSEDFGSYYCQHHYGTPFTFGSGTKLEIKR

SEQ ID NO: 11 - protein, 111 aa, Mus musculus
SQAVVTQESALTTSPGETVTLTCRSSTGAVITSNDANWVQEKPDHSFTGLIGGTNNRAPGVPARFSGSLIGDKAALTITGAQTEDEAIYFCALWYSNHWVFGGGTKLTVLG

SEQ ID NO: 12 - protein, 108 aa, Mus musculus
DIQMTQSPVSLSASVGETVTITCRASENVYSYLAWYQQKQGKSPQLLVYNAKTLAEGVPSRFSGSGSGTQFSLKINSLQPEDFGSYYCQHHFGTPLTFGAGTKLDLKR

SEQ ID NO: 13 - protein, 108 aa, Mus musculus
DIQMTQSPASLSASVGETVTITCRTSENIYSYLAWYQQKQGKSPQLLVYNAKTLAEGVPSRFSGSGSGTQFSLKINRLQPEDFGRYFCQHHYGIPFTFGSGTKLEIKR

SEQ ID NO: 14 - protein, 214 aa, Artificial Sequence
  Human frameworks, rodent CDRs
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 10 aa, Mus musculus
GYSFTAYYIQ

SEQ ID NO: 16 - protein, 10 aa, Mus musculus
GYIFSAYWMT

SEQ ID NO: 17 - protein, 10 aa, Mus musculus
GYTFTSYGIS

SEQ ID NO: 18 - protein, 10 aa, Mus musculus
GYSFTSFFIH

SEQ ID NO: 19 - protein, 10 aa, Mus musculus
GYIFITYWMT

SEQ ID NO: 20 - protein, 17 aa, Mus musculus
YISCYNGATRYNQKFKG

SEQ ID NO: 21 - protein, 17 aa, Mus musculus
QIFPVRGSADYNEIFEG

SEQ ID NO: 22 - protein, 17 aa, Mus musculus
EIYPRSVNSYYNERFKG

SEQ ID NO: 23 - protein, 17 aa, Mus musculus
WIFPGNHDVEYNEKFKG

SEQ ID NO: 24 - protein, 17 aa, Mus musculus
QIFPASGSADYNEMFEG

SEQ ID NO: 25 - protein, 17 aa, Artificial Sequence
  Rodent CDR with one amino acid substitution
QIFPASGSADYNEKFEG

SEQ ID NO: 26 - protein, 17 aa, Artificial Sequence
  Rodent CDR with four amino acid substitutions
QIFPASGSADYAQKLQG

SEQ ID NO: 27 - protein, 8 aa, Mus musculus
QGFYAMDY

SEQ ID NO: 28 - protein, 7 aa, Mus musculus
GGGGFAY

SEQ ID NO: 29 - protein, 15 aa, Mus musculus
GGNYYGRNYGDYFDY

SEQ ID NO: 30 - protein, 7 aa, Mus musculus
GGGNLPY

SEQ ID NO: 31 - protein, 7 aa, Mus musculus
GGGGFAY

SEQ ID NO: 32 - protein, 16 aa, Mus musculus
KSSQSLLDSDGKTYLN

SEQ ID NO: 33 - protein, 11 aa, Mus musculus
RASENIYSYLA

SEQ ID NO: 34 - protein, 14 aa, Mus musculus
RSSTGAVITSNDAN

SEQ ID NO: 35 - protein, 11 aa, Mus musculus
RTSENIYSYLA

SEQ ID NO: 36 - protein, 11 aa, Mus musculus
RTSENIYSYLA

SEQ ID NO: 37 - protein, 7 aa, Mus musculus
LVSKLDS

SEQ ID NO: 38 - protein, 7 aa, Mus musculus
NAKTLAE

SEQ ID NO: 39 - protein, 7 aa, Mus musculus
GTNNRAP

SEQ ID NO: 40 - protein, 7 aa, Mus musculus
NAKTLAE

SEQ ID NO: 41 - protein, 7 aa, Mus musculus
NAKTLAE

SEQ ID NO: 42 - protein, 9 aa, Mus musculus
WQGTHFPFT

SEQ ID NO: 43 - protein, 9 aa, Mus musculus
QHHYGTPFT

SEQ ID NO: 44 - protein, 9 aa, Mus musculus
ALWYSNHWV

SEQ ID NO: 45 - protein, 9 aa, Mus musculus
QHHYGIPFT

SEQ ID NO: 46 - protein, 9 aa, Mus musculus
QHHYGIPFT

SEQ ID NO: 47 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 48 - protein, 175 aa, Mus musculus
VPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 49 - DNA, 1398 bp, Artificial Sequence
  Human constant and framework regions, rodent CDRs
atggctgtgctggggctgctgttctgcctggtgacattcccaagctgtgtgctgtcccaggtgcagctggtgcagtctggcgctgaggtgaagaagcctggcgcctccgtgaaggtctcctgcaaggcttctggctacatcttcatcacctactggatgacctgggtgcggcaggcccctggccaggggctggagtggatgggccagatcttccctgccagcggctctgcagactacaacgagaagttcgaaggcagagtcaccatgaccacagacacatccaccagcacagcctacatggagctgaggagcctgagatctgacgacaccgccgtgtattactgtgccagaggcggtggcggattcgcttactggggccagggcaccctggtcaccgtctccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - DNA, 702 bp, Artificial Sequence
  Human constant and framework regions, rodent CDRs
atggctccagtgcagctgctggggctgctggtgctgttcctgccagccatgagatgtgatatccagatgacccagtctccatcctccctgtctgcctctgtgggcgacagagtgaccatcacctgcaggaccagcgagaacatctacagctacctggcctggtatcagcagaagccagggaaggcccctaagctgctgatctataacgccaagaccctggctgaaggggtgccatccaggttcagcggcagcggctctgggacagacttcaccctgaccatcagcagcctgcagcctgaggacttcgccacctactactgtcagcaccactacggaattccattcaccttcggccagggcaccaaggtggagatcaagcgtacggtggctgcaccatctgtgttcatcttccctccatctgatgagcagctgaagtctggaactgcctccgtggtgtgcctgctgaataacttctatcccagagaggccaaggtgcagtggaaggtggataacgccctccagagcggcaactcccaggagagcgtgacagagcaggacagcaaggacagcacctacagcctgagcagcaccctgaccctgagcaaagcagactacgagaaacacaaggtgtacgcctgcgaggtgacccatcagggcctgagcagccccgtgacaaagagcttcaacaggggagagtgttaa

SEQ ID NO: 51 - protein, 5 aa, Homo sapiens
TYWLG

SEQ ID NO: 52 - protein, 16 aa, Homo sapiens
IMSPVDSDIRYSPSFQ

SEQ ID NO: 53 - protein, 10 aa, Homo sapiens
RRPGQGYFDF

SEQ ID NO: 54 - protein, 11 aa, Homo sapiens
RASQGISSWLA

SEQ ID NO: 55 - protein, 7 aa, Homo sapiens
AASSLQS

SEQ ID NO: 56 - protein, 9 aa, Homo sapiens
QQYNIYPYT

SEQ ID NO: 57 - protein, 119 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 58 - protein, 108 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 59 - protein, 449 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 9 aa, Homo sapiens
FTFSSYGMH

SEQ ID NO: 62 - protein, 17 aa, Homo sapiens
FIRYDGSNKYYADSVKG

SEQ ID NO: 63 - protein, 6 aa, Homo sapiens
HGSHDN

SEQ ID NO: 64 - protein, 13 aa, Homo sapiens
SGSRSNIGSNTVK

SEQ ID NO: 65 - protein, 7 aa, Homo sapiens
YNDQRPS

SEQ ID NO: 66 - protein, 12 aa, Homo sapiens
QSYDRYTHPALL

SEQ ID NO: 67 - protein, 115 aa, Homo sapiens [sequence omitted]

SEQ ID NO: 68 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 69 - protein, 445 aa, Homo sapiens
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 217 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - DNA, 61 bp, Homo sapiens
tcagcctgaagaagggctaatggccgaaaakattttatgtgagtttcacatgggggcatat

SEQ ID NO: 72 - DNA, 61 bp, Homo sapiens
tcagcctgaagaagggctaatggccgaaaagattttatgtgagtttcacatgggggcatat

SEQ ID NO: 73 - DNA, 61 bp, Homo sapiens
tcagcctgaagaagggctaatggccgaaaatattttatgtgagtttcacatgggggcatat

SEQ ID NO: 74 - DNA, 61 bp, Homo sapiens
aaacaatgtaaactcgaataaggataagcaktccaacagattgaaaaccaagaaatacatt

SEQ ID NO: 75 - DNA, 61 bp, Homo sapiens
aaacaatgtaaactcgaataaggataagcagtccaacagattgaaaaccaagaaatacatt

SEQ ID NO: 76 - DNA, 61 bp, Homo sapiens
aaacaatgtaaactcgaataaggataagcattccaacagattgaaaaccaagaaatacatt

SEQ ID NO: 77 - DNA, 61 bp, Homo sapiens
atattgaagcagttaagaagacccaacacayggataatatgaatacctgaagaagaaaacc

SEQ ID NO: 78 - DNA, 61 bp, Homo sapiens
atattgaagcagttaagaagacccaacacacggataatatgaatacctgaagaagaaaacc

SEQ ID NO: 79 - DNA, 61 bp, Homo sapiens
atattgaagcagttaagaagacccaacacatggataatatgaatacctgaagaagaaaacc

SEQ ID NO: 80 - DNA, 61 bp, Homo sapiens
gagatttcttgctaattttctagatcagaamcatttcttttctttgtctttttttttgtcc

SEQ ID NO: 81 - DNA, 61 bp, Homo sapiens
gagatttcttgctaattttctagatcagaaacatttcttttctttgtctttttttttgtcc

SEQ ID NO: 82 - DNA, 61 bp, Homo sapiens
gagatttcttgctaattttctagatcagaaccatttcttttctttgtctttttttttgtcc

SEQ ID NO: 83 - DNA, 61 bp, Homo sapiens
gccactgactagtcatgtgacctcagatacrtcattttgtgtcctcacctgttcaacaagg

SEQ ID NO: 84 - DNA, 61 bp, Homo sapiens
gccactgactagtcatgtgacctcagatacgtcattttgtgtcctcacctgttcaacaagg

SEQ ID NO: 85 - DNA, 61 bp, Homo sapiens
gccactgactagtcatgtgacctcagatacatcattttgtgtcctcacctgttcaacaagg